(19) United States
(12) Patent Application Publication
(10) Pub. No.: US 2022/0133463 A1
(43) Pub. Date: May 5, 2022

(54) MODULAR BRANCHED ENDOPROSTHETIC SYSTEMS, DEVICES, AND METHODS

(71) Applicant: W. L. Gore & Associates, Inc., Newark, DE (US)

(72) Inventor: Frederick S. Korte, Flagstaff, AZ (US)

(21) Appl. No.: 17/434,083

(22) PCT Filed: Feb. 26, 2019
(86) PCT No.: PCT/US2020/019916

§ 371 Date: Aug. 26, 2021

Related U.S. Application Data
(60) Provisional application No. 62/810,736, filed on Feb. 26, 2019.

(51) Int. Cl. A61F 2/07; A61F 2/954; A61F 2/852; A61F 2/89

(52) U.S. Cl. CPC A61F 2/07; A61F 2/954; A61F 2002/061; A61F 2/89; A61F 2/852

(57) ABSTRACT

Devices, systems and methods of endoluminally delivering a modular endoprosthetic system in accordance with various embodiments are disclosed herein for treating disease of human vasculature. In various embodiments, the modular endoprosthetic system includes a plurality of expandable endoprosthesis components that are coupled together to define the modular endoprosthetic system, wherein the modular endoprosthetic system provides for retrograde perfusion of a branch vessel from a main vessel.

### CROSS-REFERENCE TO RELATED APPLICATION

This application is a national phase application of PCT Application No. PCT/US2020/019916, internationally filed on Feb. 26, 2020, which claims the benefit of Provisional Application No. 62/810,736, filed Feb. 26, 2019, which are incorporated herein by reference in their entireties for all purposes.

### FIELD

The present disclosure relates to delivery systems and methods of endoluminally delivering modular branched vascular endoprosthetic systems to vascular treatment sites.

### BACKGROUND

There is a need for advanced devices, tools, systems and methods used for the endoluminal treatment of vascular diseases in regions of branch vessels and main vessel junctions, including diseases affecting the aorta, including the descending aorta adjacent to the celiac artery, superior mesenteric artery and the two renal arteries.

### SUMMARY

According to one example, (“Example 1”), a method includes providing a first expandable device configured to be deployed in a main vessel; providing a second expandable device configured to interface with the first expandable device, wherein the second expandable device includes a portal therein; providing a branch vessel expandable device configured once expanded to form a fluid connection between a branch vessel and the second expandable device through the portal; placing a branch guidewire into the branch vessel; positioning the branch vessel expandable device over the branch guidewire into the branch vessel while maintained in a not fully deployed state; placing and deploying the first expandable device in the main vessel, wherein the branch vessel expandable device is positioned exterior to the first expandable device; placing and deploying the second expandable device downstream to the branch vessel, wherein the branch guidewire and the branch vessel expandable device each extend through the portal of the second expandable device; and deploying the branch vessel expandable device such that the branch vessel expandable device is fluidly coupled with the second expandable device via the portal of the second expandable device, wherein blood is perfused into the branch vessel through retrograde flow.

According to another example, (“Example 2”), further to Example 1, the branch guidewire is placed into a renal artery, and wherein the branch vessel expandable device is placed over the branch guidewire into the renal artery while maintained in the not fully deployed state, and wherein the first expandable device is placed and deployed in an aorta of a patient with the branch vessel expandable device positioned exterior to the first expandable device, and wherein the second expandable device is placed and deployed at least partially downstream to the renal artery with the branch guidewire and the branch expandable device extending through the portal to form a fluid connection between the renal artery and the second expandable device to provide for retrograde perfusion of blood to the renal artery.

According to another example, (“Example 3”), further to Example 1, the main vessel is a common iliac artery and wherein the branch vessel is an internal iliac artery.

According to another example, (“Example 4”), further to Example 1, the main vessel is an external iliac artery and wherein the branch vessel is a femoral artery.

According to another example, (“Example 5”), further to any of Examples, positioning the branch vessel expandable device over the branch guidewire into the branch vessel includes advancing the branch vessel expandable device over the branch guidewire after the second expandable device is deployed.

According to another example, (“Example 6”), further to Example 5, the branch vessel expandable device is deployed after the second expandable device is deployed.

According to another example, (“Example 7”), further to any of Examples, the branch vessel expandable device is directly coupled to the second expandable device.

According to another example, (“Example 8”), a method includes providing a first expandable device configured to be deployed in a blood vessel; providing a second expandable device configured to interface with the first expandable device, wherein the second expandable device includes a portal therein; providing a branch expandable device configured to form a fluid connection between a branch vessel and the second expandable device through the portal; placing a branch guidewire into the branch vessel; placing and deploying the first expandable device in the main vessel; placing and deploying the second expandable device downstream from the branch vessel, wherein the branch guidewire extends through the portal; positioning the branch expandable device over the branch guidewire to interconnect the branch vessel and the second expandable device exterior to the first expandable device; and deploying the branch expandable device to form a fluid connection between the branch vessel and the second expandable device, wherein blood is perfused into the branch vessel through retrograde flow.

According to another example, (“Example 9”), further to Example 8, the branch guidewire is placed into a renal artery, and wherein the second expandable device is placed and deployed at least partially downstream to the renal artery with the second expandable device fluidly coupled with the renal artery via the branch expandable device to provide for retrograde perfusion of blood to the renal artery.

According to another example, (“Example 10”), further to Example 8, the main vessel is a common iliac artery and wherein the branch vessel is an internal iliac artery.

According to another example, (“Example 11”), further to Example 8, the main vessel is an external iliac artery and wherein the branch vessel is a femoral artery.

According to another example, (“Example 12”), further to any of Examples 8 to 11, the method further includes deploying a third expandable device between the second expandable device and the branch vessel expandable device.

According to another example, (“Example 13”), further to Example 12, the third expandable device is advanced into position over the branch guidewire.

According to another example, (“Example 14”), further to any of Examples 12 and 13, the third expandable device is deployed after the branch vessel expandable device is deployed and after the second expandable device is deployed.

According to another example, (“Example 15”), further to any of the preceding Examples, the second expandable device is provided in a collapsed delivery configuration with a removable guidewire tube extending through the portal to allow for insertion of the branch guidewire therethrough.

According to another example, (“Example 16”), further to Example 15, the method further includes removing the removable guidewire tube after insertion of the second guidewire through the removable guidewire tube.

According to another example, (“Example 17”), further to Example 16, the method further includes removing the removable guidewire tube prior to insertion of the second expandable device into the main vessel.

According to another example, (“Example 18”), further to any of the preceding Examples, the first and second expandable devices are deployed prior to deploying the branch vessel expandable device.

According to another example, (“Example 19”), further to any of Examples 8 to 17, the branch vessel expandable device is deployed prior to the second expandable device being deployed.

According to another example, (“Example 20”), further to any of Examples 1 to 18, the branch vessel expandable device is deployed after the second expandable device is deployed.

According to another example, (“Example 21”), further to any of the preceding Examples, the second expandable device is deployed after the first expandable device is deployed.

According to another example, (“Example 22”), further to any of the preceding Examples, each of the first expandable device, the second expandable device, and the branch expandable device are advanced from a first access site that is downstream from the branch vessel.

According to another example, (“Example 23”), further to any of the preceding Examples, the branch vessel expandable device is fluidly coupled to the second expandable device via the portal.

According to another example, (“Example 24”), further to any of the preceding Examples, the first expandable device is advanced over a first guidewire separate distinct from the branch guidewire.

According to another example, (“Example 25”), further to Example 24, the second expandable device is advanced over each of the first guidewire and the branch guidewire.

According to another example, (“Example 26”), further to any of the preceding Examples, the portal is positioned in a sidewall of the second expandable device.

According to another example, (“Example 27”), an expandable device configured to repair a main vessel extending from an upstream end to a downstream end includes: a first expandable device configured to be deployed in a blood vessel; a second expandable device configured to interface with the first expandable device and including a portal in a sidewall of the second expandable device; and a branch vessel expandable device configured to form a fluid connection between a branch vessel and the second expandable device by extending through the portal, wherein the branch expandable device is configured to have sufficient length to allow for retrograde perfusion to the branch vessel through the branch vessel expandable device in association with the second expandable device being implanted downstream from the branch vessel.

According to another example, (“Example 28”), further to Example 27, the branch vessel expandable device is configured with sufficient radial expansion force to maintain significant flow therethrough when deployed exterior to the first expandable device between the first expandable device and a wall of the main vessel.

According to another example, (“Example 29”), further to any of Examples 27 to 28, the branch vessel expandable device is directly coupled to the second expandable device.

According to another example, (“Example 30”), further to any of Examples 27 to 28, the device further includes a third expandable device extending between the second expandable device and the branch vessel expandable device and configured to allow for retrograde perfusion to the branch vessel through the branch vessel expandable device and the third expandable device.

According to another example, (“Example 31”), further to any of Examples 27 to 30, the side wall of the second expandable device includes a recessed portion that is recessed relative to the side wall, the portal being located in the recessed portion.

According to another example, (“Example 32”), further to any of Examples 27 to 30, the device further including a downstream expandable device extending from the second expandable component to fluidly couple the second expandable component to one or more vessels downstream of the second expandable component.

According to another example, (“Example 33”), further to any of Examples 27 to 30, the first expandable device includes a body portion, a first leg and a second leg branching from the body portion, and the second expandable device is configured to interface with one of the first leg and the second leg of the first expandable device.

According to another example, (“Example 34”), further to Example 33, the first leg and the second leg are structurally biased to angle apart from one another.

According to another example, (“Example 35”), further to any of Examples 33 to 34, the second expandable device is configured to interface with the first second leg of the first expandable device, and further comprising an additional second expandable device configured to interface with the second leg of the first expandable device and including a portal in a sidewall of the second expandable device.

According to another example, (“Example 36”), further of Example 35, the device also includes an additional branch vessel expandable device configured to form a fluid connection between a second branch vessel and the additional second expandable device by extending through the portal.

According to another example, (“Example 37”), further to any of Examples 27 to 36, the second expandable component includes a proximal end and a distal end, and a tapered configuration with the proximal end having a diameter less than the distal end.

According to another example, (“Example 38”), further to any of Examples 27 to 37, the portal of the second expandable device is an aperture in the sidewall of the second expandable device.

According to another example, (“Example 39”), further to any of Examples 27 to 38, the device also includes a bridge expandable component configured to position between the second expandable device configured and the first expandable device.

According to another example, (“Example 40”), further of Example 39, the bridge expandable component is configured to deploy with a first end coupled with the portal of the second expandable component and with a second end coupled with the second expandable device component.

According to another example, (“Example 41”), further to any of Example 40, the branch vessel expandable component includes one or more tissue anchors for engaging tissue.

While multiple embodiments are disclosed, still other embodiments will become apparent to those skilled in the art from the following detailed description, which shows and describes illustrative examples. Accordingly, the drawings and detailed description are to be regarded as illustrative in nature and not restrictive.

### BRIEF DESCRIPTION OF THE DRAWINGS

The features and advantages of the present disclosure will become more apparent from the detailed description set forth below when taken in conjunction with the drawings.

FIG. 1 is a cross-sectional representation of a human anatomy showing an aorta, including two renal arteries and two iliac arteries.

FIG. 2 is a cross-sectional representation of the human anatomy of FIG. 1 showing a modular endoprosthetic system implanted therein, according to some embodiments.

FIG. 3 illustrates a main vessel expandable component in the form of a branched expandable endoprosthesis, according to some embodiments.

FIG. 4 illustrates a branch vessel expandable component, according to some embodiments.

FIG. 5A illustrates a portal expandable component, according to some embodiments.

FIG. 5B illustrates the portal expandable component of FIG. 5A with a branch vessel expandable component coupled therewith, according to some embodiments.

FIG. 6 illustrates a portal expandable component having removable guidewire tubes extending therethrough, according to some embodiments.

FIG. 7 illustrates the portal expandable component of FIG. 6 in a constrained and collapsed delivery configuration, according to some embodiments.

FIG. 8 illustrates a method of delivering a modular endoprosthetic system, according to some embodiments.

FIG. 9A is a cross-sectional representation of the human anatomy with a guidewire extending through a lower access site and traversing a main vessel and extending into a branch vessel.

FIG. 9B is a detailed view of the cross-sectional representation of the human anatomy of FIG. 9A showing a branch vessel expandable device in a constrained and collapsed delivery configuration positioned within the branch vessel, according to some embodiments.

FIG. 9C is the detailed view of the cross-sectional representation of the human anatomy of FIG. 9B with a second guidewire positioned within the human anatomy in the region of the branch vessel expandable device, according to some embodiments.

FIG. 9D is the cross-sectional representation of the human anatomy of FIG. 9A with a main vessel expandable device fully deployed within the main vessel, according to some embodiments.

FIG. 9E is the cross-sectional representation of the human anatomy of FIG. 9D with additional expandable components deployed within the anatomy, according to some embodiments.

FIG. 10 illustrates a subset of steps corresponding to the method of delivering a modular endoprosthetic system of FIG. 8, according to some embodiments.

FIG. 11 is a cross-sectional representation of the human anatomy with a main vessel expandable device and a portal expandable device deployed in the main vessel and with a branch vessel expandable device deployed in the branch vessel, according to some embodiments.

FIG. 12 is the cross-sectional representation of the human anatomy of FIG. 11 showing a guidewire extending through the portal expandable component and the branch vessel expandable component, according to some embodiments.

FIG. 13 is the cross-sectional representation of FIG. 12 of the human anatomy with a bridge expandable component deployed and extending between the portal expandable component and the branch vessel expandable component, according to some embodiments.

FIG. 14 illustrates a method of delivering a modular endoprosthetic system, according to some embodiments.

FIG. 15 is a cross-sectional representation of the human anatomy with multiple guidewires extending through a lower access site and extending into the anatomy, according to some embodiments.

FIG. 16 is the cross-sectional representation of the human anatomy of FIG. 15 showing a main vessel expandable device deployed in the main vessel, according to some embodiments.

FIG. 17 is the cross-sectional representation of the human anatomy of FIG. 16 with a portal expandable component in a constrained and collapsed delivery configuration being advanced along a guidewire toward a desired position within the human anatomy, according to some embodiments.

FIG. 18 is the cross-sectional representation of the human anatomy of FIG. 17 with the portal expandable component deployed, according to some embodiments.

FIG. 19 is the cross-sectional representation of the human anatomy of FIG. 18 with a branch vessel expandable component being advanced in a constrained and collapsed delivery configuration, according to some embodiments.

FIG. 20 is the cross-sectional representation of the human anatomy of FIG. 19 with the main vessel expandable component, the portal expandable component, and the branch vessel expandable component fully expanded, according to some embodiments.

### DETAILED DESCRIPTION

Persons skilled in the art will readily appreciate that various aspects of the present disclosure can be realized through various methods and apparatuses configured to perform the intended functions. It should also be noted that the accompanying drawing figures referred to herein are not all drawn to scale, but can be exaggerated to illustrate various aspects of the present disclosure, and in that regard, the drawing figures should not be construed as limiting. Finally, although the present disclosure can be described in connection with various principles and beliefs, the present disclosure should not be bound by theory.

Throughout this specification and in the claims, the term “distal” refers to a location that is, or a portion of an endoluminal device (such as a stent-graft) that when implanted is, further downstream with respect to blood flow than another portion of the device. Similarly, the term “distally” refers to the direction of blood flow or further downstream in the direction of blood flow.

The term “proximal” refers to a location that is, or a portion of an endoluminal device that when implanted is, further upstream with respect to blood flow than another portion of the device. Similarly, the term “proximally” refers to the direction opposite to the direction of blood flow or upstream from the direction of blood flow.

With further regard to the terms proximal and distal, and because the present disclosure is not limited to peripheral and/or central approaches, this disclosure should not be narrowly construed with respect to these terms. Rather, the devices and methods described herein can be altered and/or adjusted relative to the anatomy of a patient.

In some embodiments, the devices and systems described herein may be configured to be used in a retrograde manner, i.e. delivered to a target site in a direction opposite to that of blood flow, or in an antegrade manner, i.e. the device is delivered to a target site in the direction of blood flow.

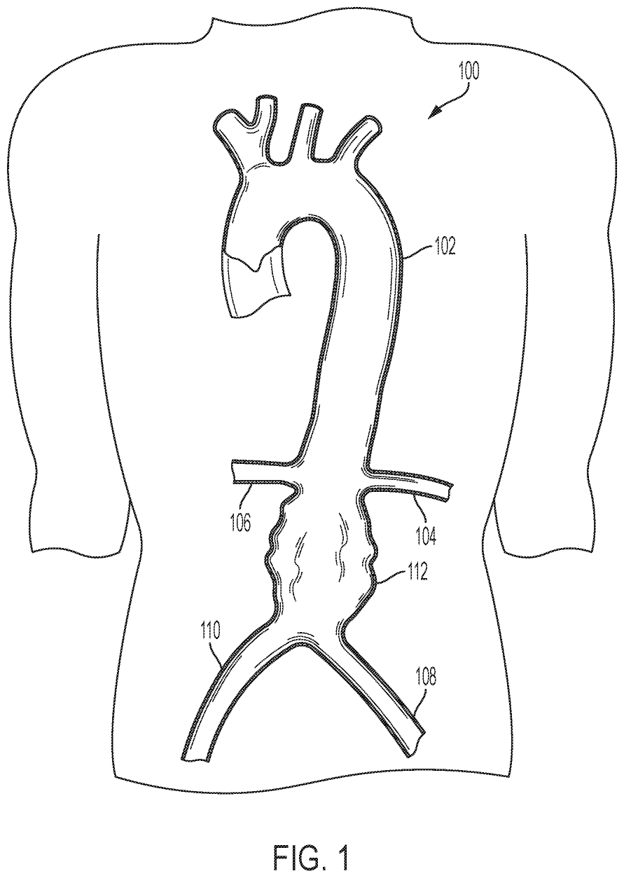

Devices, systems and methods of endoluminally delivering a modular endoprosthetic system in accordance with various embodiments are disclosed herein for treating disease of human vasculature. In various embodiments, the modular endoprosthetic system includes a plurality of expandable endoprosthesis components that are coupled together to define the modular endoprosthetic system, as described further below. FIG. 1 shows a vasculature including an aorta 100 having, a descending aorta 102, renal arteries 104 and 106, and iliac arteries 108 and 110. The aorta 100 includes an abdominal aortic aneurysm 112 downstream to the renal arteries 104 and 106. It will be appreciated that the location and configuration of the abdominal aortic aneurysm 112 is not intended to limit the scope of the disclosure or the applicability of the modular endoprosthetic system described herein. The modular endoprosthetic systems described herein have wide applicability to vascular diseases involving the treatment of main and branch vessels.

Thus, although the description and figures of the present application are illustrated in the context of treating the aorta 100, including the descending aorta 102 shown in FIG. 1, it is to be appreciated that the devices, systems, and methods of the present disclosure may be applied to treat other portions of the vasculature, including, for example, any disease where a larger vessel and one or more branch vessels are to be treated. Such treatment may include treatments involving regions within the vasculature that include one or more branch vessels, and thus may require main vessel endoprosthesis components including bifurcated or non-bifurcated configurations.

In various embodiments, the modular endoprosthetic system of the present disclosure includes a plurality of expandable endoprosthesis components, such as stents and stent grafts, that are assembled together to collectively form or otherwise define the modular endoprosthesis. That is, in various examples, the modular endoprosthesis includes a plurality of distinct and independent expandable endoprosthetic components that are configured to interface with other distinct and independent expandable endoprosthetic components. The modular configuration provides for versatility on how and where the modular endoprosthesis can be employed, and in what configuration it is employed.

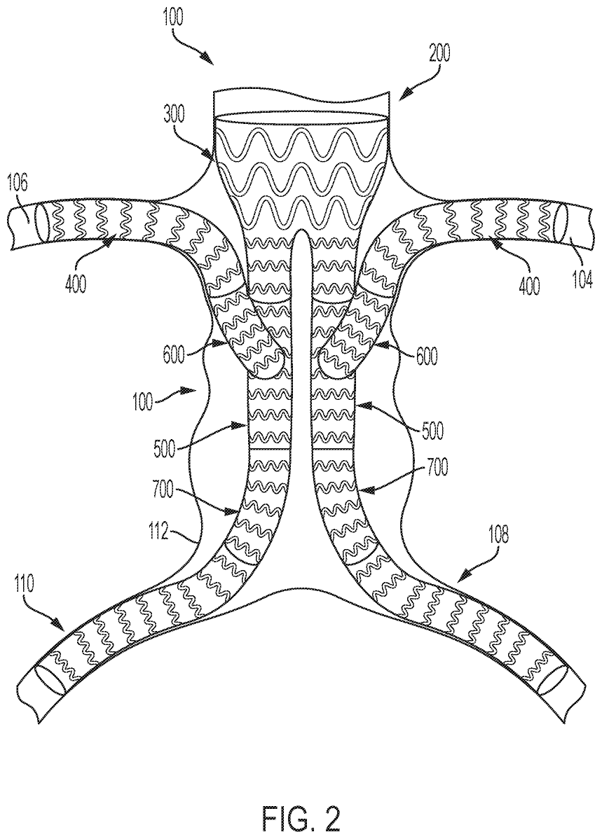

Referring to FIG. 2, for example, a modular endoprosthetic system 200 is shown implanted within a vasculature. As show, the modular endoprosthetic system 200 includes a first or main vessel expandable component 300, a branch vessel expandable component 400, and a second or portal expandable component 500. When fully assembled and deployed, each of the main vessel expandable component 300, the branch vessel expandable component 400, and the portal expandable component 500 are fluidly coupled together. As such, blood entering the main vessel expandable component 300 can perfuse to the branch vessel component 400 via the portal expandable component 500. As shown, the blood entering the main vessel expandable component 300 propagates through the main vessel expandable component 300 and the portal expandable component 500 via antegrade flow, and propagates into the branch vessels 104 and 106 via retrograde flow through the branch vessel component 400. This is because blood flow exiting the portal expandable component 500 and flowing toward the branch vessels 104 and 106 exits the portal expandable component 500 downstream of the branch vessels 104 and 106. In various examples, as discussed further below, each and every one of the various expandable components collectively defining the modular endoprosthetic system 200 can be delivered to the treatment site within the vasculature from the same access site, which may be an upper or lower access site relative to the treatment region. As such, it is to be appreciated that each and every one of the various expandable components collectively defining the modular endoprosthetic system 200 can be delivered and deployed at the treatment site via retrograde delivery (e.g., through a femoral access).

Also illustrated in FIG. 2 are optional bridge expandable components 600, which are not essential, as mentioned further below, as well as one or more downstream expandable components 700.

In various examples, the modular endoprosthetic system 200 is configured such that assembly and/or deployment of the various components of may occur in-situ. Thus, assembly and/or deployment of the various components of the modular endoprosthetic system 200 may include sequenced delivery and deployment of the various components in lieu of a single non-modular deployment sequence. As such, one or more expandable components of the modular endoprosthetic system 200 may be delivered and fully deployed within the vasculature prior to another one of the expandable components being inserted into the vasculature or delivered to the treatment site.

The modular expandable components of the present disclosure may include one or more modular stent or stent graft components, and thus may generally include one or more of a support component or element and a graft component or element, as discussed further below. These expandable components (also referred to as modular stent and/or stent graft components) may be configured to dilate from a delivery configuration, through a range of larger intermediary configurations, and toward a deployed configuration. The expandable components may be configured to engaged with one another and/or one or more portions of the vasculature, such as the vessel wall at a treatment site. The expandable components can have various configurations such as, for example, rings, cut tubes, wound wires (or ribbons) or flat patterned sheets rolled into a tubular form. In some examples, the stent and/or stent graft components may include metallic, polymeric or natural materials and can comprise conventional medical grade materials such as nylon, polyacrylamide, polycarbonate, polyethylene, polyformaldehyde, polymethylmethacrylate, polypropylene, polytetrafluoroethylene, polytrifluorochlorethylene, polyvinylchloride, polyurethane, elastomeric organosilicon polymers; metals such as stainless steels, cobalt-chromium alloys and nitinol and biologically derived materials such as bovine arteries/veins, pericardium and collagen. In some examples, the stent and/or stent graft components may include bioresorbable materials such as poly(amino acids), poly(anhydrides), poly(caprolactones), poly(lactic/glycolic acid) polymers, poly(hydroxybutyrates) and poly(orthoesters).

In various embodiments, potential non-limiting materials for graft elements include, for example, expanded polytetrafluoroethylene (ePTFE), polyester, polyurethane, fluoropolymers, such as perfluoroelastomers and the like, polytetrafluoroethylene, silicones, urethanes, ultra high molecular weight polyethylene, aramid fibers, and combinations thereof. Graft element material may additionally or alternatively include high strength polymer fibers such as ultra high molecular weight polyethylene fibers (e.g., Spectra®, Dyneema Purity®, etc.) or aramid fibers (e.g., Technora®, etc.). Any graft element that can be delivered via a catheter is in accordance with the present disclosure. In some examples, the graft element may include a bioactive agent. In some examples, an ePTFE graft includes a carbon component along a blood contacting surface thereof. Further detail of materials and general construction of stents, graft elements and stent grafts are generally disclosed in U.S. Pat. Nos. 6,042,605; 6,361,637; and 6,520,986 all to Martin et al.

In various embodiments, a support component and/or graft element can comprise a therapeutic coating. In these embodiments, the interior and/or exterior of the support component and/or graft element can be coated with, for example, a CD34 antigen. Additionally, any number of drugs or therapeutic agents can be used to coat the graft element, including, for example heparin, sirolimus, paclitaxel, everolimus, ABT-578, mycophenolic acid, tacrolimus, estradiol, oxygen free radical scavenger, biolimus A9, anti-CD34 antibodies, PDGF receptor blockers, MMP-1 receptor blockers, VEGF, G-CSF, HMG-CoA reductase inhibitors, stimulators of iNOS and eNOS, ACE inhibitors, ARBs, doxycycline, and thalidomide, among others.

Consistent with the description above regarding the versatility of the devices, systems, and methods described herein, while the modular endoprosthetic system 200 shown in FIG. 2 includes a bifurcated main vessel expandable component 300, non-bifurcated expandable components may be used in applications where bifurcated components are not necessary. For instance, non-bifurcated main vessel expandable components may be used in applications where dedicated perfusion from the main vessel component to multiple branch vessels is not required. An “AUI” (Aorto-uni-iliac) treatment generally involves supplying blood to only one of the iliac arteries directly from the abdominal aorta because the second of the iliac arteries may be either already occluded or declared unsalvageable by the physician. In some instances, the physician may employ the portal expandable component 500 in combination with a main vessel expandable component 300 and a branch vessel expandable component 400, for example, while effectively not perfusing the other of the iliac arties directly from the abdominal aorta. Perfusion of the second of the iliac arteries is instead accomplished by a surgical bypass graft extending directly between the patients external iliac or femoral arteries.

As shown in FIG. 2, in various embodiments, the modular endoprosthetic system 200 may include additional expandable components based on the needs of the anatomy and the particular vascular treatment being performed. The modular endoprosthetic system 200, for instance, includes an optional bridge expandable endoprosthesis 208 that is configured to extend between and fluidly couple together the portal expandable component 500 and the branch vessel expandable component 400. In some examples, a bridge expandable endoprosthesis 600 may be utilized to increase a distance between the portal expandable component 500 and the branch vessel expandable component 400, which may be required based on the particular makeup of the patient's anatomy, and/or may be required as a result of the particular delivery sequence employed. The modular endoprosthetic system 200 shown in FIG. 2 also includes downstream expandable components 700, one or more of which may be optionally employed to fluidly couple the portal expandable component 500 to one or more vessels downstream of the portal expandable component 500.

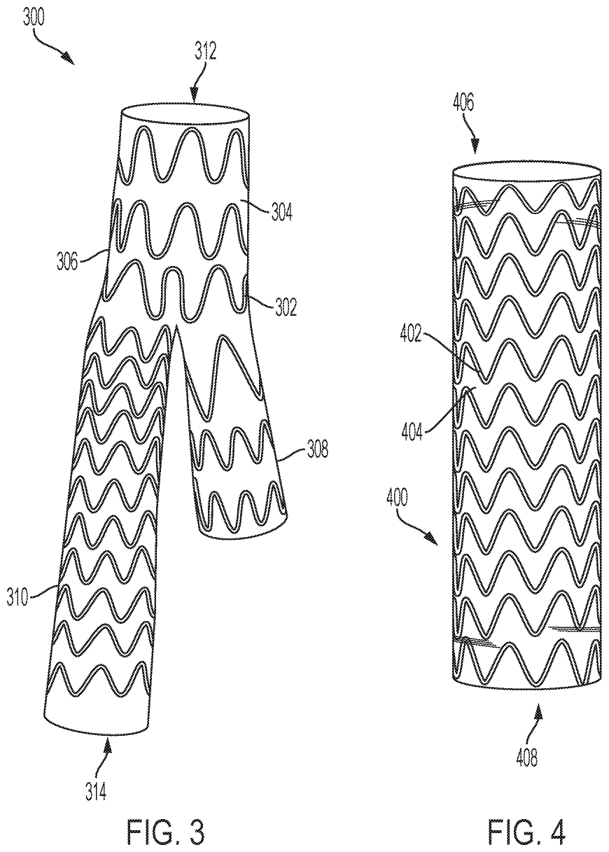

FIGS. 3 to 5 show some of the various expandable components of the modular endoprosthetic system 200. For example, FIG. 3 illustrates the main vessel expandable component 300 including a support component 302 and a graft component 304. Main vessel expandable component 300 is shown in FIGS. 2 and 3 as a branched endoprosthesis, although non-branched endoprosthesis configurations are also contemplated, as mentioned above. The main vessel expandable component 300 includes a body portion 306 (also referred to as a trunk portion), a contralateral leg 308 (also referred to herein as a first leg or a second leg), and an ipsilateral leg 310 (also referred to herein as a second leg or a first leg, depending on the reference made to the contralateral leg 308). In various examples, the main vessel expandable component 300 provides a collapsed delivery configuration for endoluminal delivery and an expanded configuration larger than collapsed delivery configuration.

In various examples, the graft component 304, is generally any abluminal (i.e., outer, vessel surface) or luminal (i.e., inner, blood flow surface) covering configured to partially or substantially cover one or more support components. In various embodiments, a graft component, such as graft component 304, comprises ePTFE. However, other useful materials for the graft component may comprise one or more of nylons, polycarbonates, polyethylenes, polypropylenes, polytetrafluoroethylenes, polyvinyl chlorides, polyurethanes, polysiloxanes, and other biocompatible materials, or any of the other graft element materials mentioned above.

In various examples, the graft component 304 is fixedly secured or otherwise coupled at a single or a plurality of locations to the abluminal or luminal surface of the support component, for example, using one or more of taping, heat shrinking, adhesion and other processes known in the art. In some embodiments, a plurality of graft components are used and may be coupled to both the abluminal and luminal surfaces of the support component(s). In other embodiments, a plurality of graft components “sandwich” the support component(s), the graft components being attached to each other within voids of the support components.

In various embodiments, the support component 302 (also referred to as a stent component) provides structural support for the graft component 304 of the main vessel expandable component and/or the vasculature to be treated. Support component 302, may be a stent comprised of a wire including a helical configuration or may be comprised of one or a plurality of rings. Among other configurations, the wire or a ring itself may be linear or have a sinusoidal or zig-zag pattern. In some examples, the support component 302 may be cut from a tube and have any pattern suitable for the treatment.

The support component 302 can be comprised of a shape-memory material, such as nitinol. In other embodiments, however, the support component 302 may be comprised of other materials, self-expandable or otherwise expandable (e.g., with a conventional balloon catheter or spring mechanism), such as various metals (e.g., stainless steel), alloys and polymers.

In various examples, the cross-sections of one or more of the body portion 306, and the first and second legs 308 and 310 may be circular, ovoidal, or have polygonal features with or without curved features. These cross-sectional shapes may also be either substantially constant or variable along their respective axial lengths. For instance, in an embodiment of a bifurcated endoprosthesis, a cross-section of the body portion 306 may be substantially circular at its distal end but taper to have an ovoidal rectangular cross-section with a smaller cross-sectional surface area in its bifurcation region adjacent the first and second legs 308 and 310.

The first and second legs 308 and 310 are shown as generally branching off of and in luminal communication with the body portion 306. As shown, each of the first and second legs includes a first end that is connected to or otherwise integral with an end of body portion 306, and a second end that extends away from the body portion 306 and the first end. The first and second ends 308 and 310 may also be structurally biased to angle apart from one another, such as in a Y configuration, so as to face or direct them toward their respective vessels to be treated. The structural bias may arise from either or both of graft component 304 and support component 302. Additionally, the axial length of first and second legs 308 and 310 may be the same or may be different, as shown. In various examples, an end of the body portion 306 opposite the end of the body portion 306 from which the first and second legs 308 and 310 are coupled defines a proximal end 312 of the main vessel expandable component 300, while one or more of the ends of the first and second legs 308 and 310 opposite the ends of the first and second legs 308 and 310 coupled to the body portion 306 defines a distal end 314 of the main vessel expandable component 300. In some examples, the proximal end 312 of the main vessel expandable component 300 is configured to anchor against or to the vasculature, such as a vessel wall, while the distal end 314 is configured to interface with one or more other expandable components, as discussed further below. In some examples, the proximal end 312 may be configured to interface with one or more other expandable components. Suitable examples of main vessel expandable components, including branch vessel expandable components, can be found in U.S. Pat. Nos. 7,682,380, 8,474,120, 8,945,200, 8,267,988 and 9,827,118.

As shown in FIG. 4, the branch vessel expandable component 400 generally includes a support component 402 and a graft component 404. The branch vessel expandable component 400 includes a proximal end 406 and a distal end 408, and may have a tapered or non-tapered configuration. The proximal and distal ends 406 and 408 may be configured to interface with one or more other expandable components of the modular endoprosthetic system 200. In some examples, one or more other expandable components of the modular endoprosthetic system 200 may be deployed within a lumen of the branch vessel expandable component 400 at the proximal and/or distal ends 406 and 408. Thus, in some examples, the branch vessel expandable component 400 may be configured to accommodate the deployment of another expandable component within the lumen of the branch vessel expandable component 400 at the proximal and/or distal ends 406 and 408. Additionally or alternatively, the branch vessel expandable component 400 may be configured such that one or more of its proximal and distal ends 406 and 408 can be deployed within a lumen of another expandable component of the modular endoprosthetic system 200.

The support component 402 and the graft component 404 may be of similar constructions to the support and graft components 302 and 304 mentioned above, and may also be coupled to one another in any suitable manner known in the art, including those manners mentioned above. In various examples, the branch vessel expandable component 400 provides a collapsed delivery configuration for endoluminal delivery and an expanded deployed configuration larger (e.g., larger in diameter and/or length) than collapsed delivery configuration. Suitable examples of branch vessel expandable components can be found in U.S. Publication No. US2016/0143759 to Bohn et al., filed Nov. 24, 2015, and titled “BALLOON EXPANDABLE ENDOPROSTHESIS.”

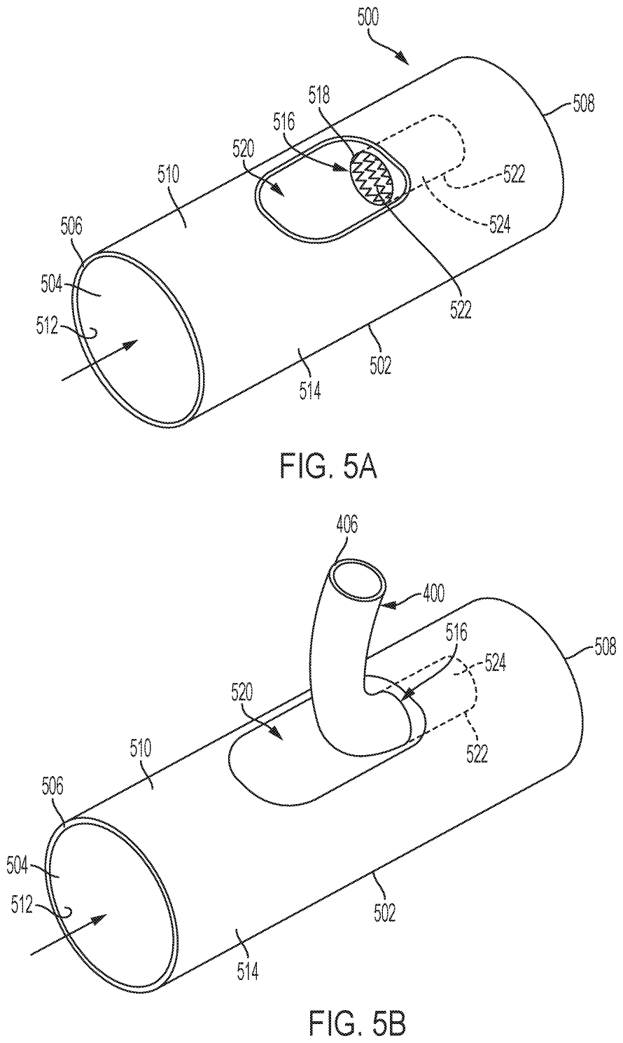

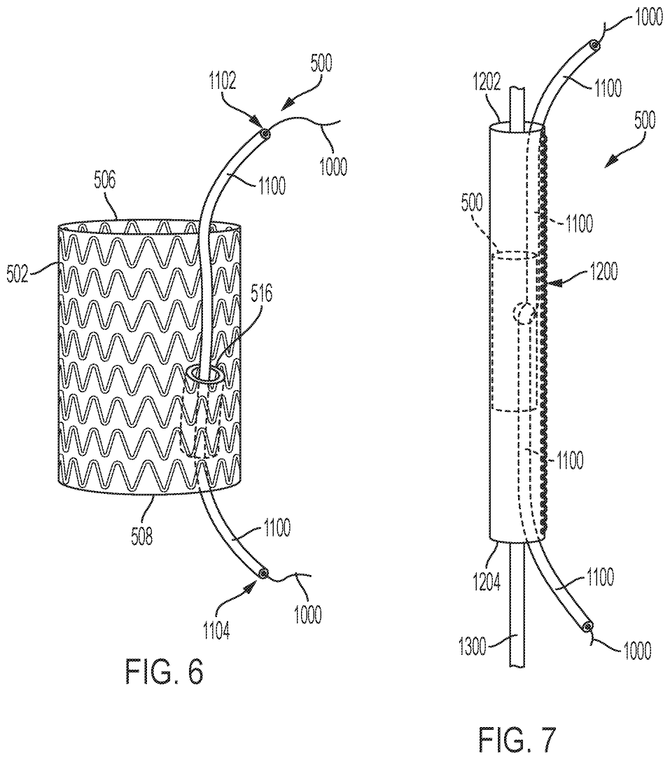

As shown in FIG. 5A, the portal expandable component 500 includes a main body 502 having a main lumen 504. The main body 502 of the portal expandable component 500 has opposite first and second ends, 506 and 508, and a wall 510 extending generally longitudinally between the first and second ends 506 and 508. The portal expandable component can be tapered or non-tapered. The wall 510 has an internal surface 512 that defines the main lumen 504. The wall 510 also has an outer surface 514 opposite the internal surface 512. Consistent with the discussion above regarding the main vessel expandable component 300 and the branch vessel expandable component 400, the portal expandable component 500 may comprise a support component and a graft component (not shown for clarity purposes, see, e.g., FIG. 6). In some examples, the wall 510 is defined by one or more of the support and graft components of the portal expandable component 500. The support and graft components of the portal expandable component 500 may therefore be of similar constructions to the support and graft components of the main vessel expandable component 300 and the branch vessel expandable component 400, mentioned above, and may also be coupled to one another in any suitable manner, including those mentioned above. In various examples, the portal expandable component 500 provides a collapsed delivery configuration for endoluminal delivery and an expanded configuration larger than collapsed delivery configuration. Additionally, like the branch vessel expandable component 400, the portal expandable component 500 may be configured to interface with one or more other expandable components at its first and second ends 506 and 508.

In various examples, the portal expandable component 500 includes at least one portal 516 situated along the wall 510 between the first and second ends 506 and 508 of the portal expandable component 500. In various examples, the portal 516 is defined as an opening 518 in the wall 510 exposing the lumen 504. As such, the portal 516 provides an access to the lumen 504 of the portal expandable component 500 such that one or more auxiliary expandable components of the modular endoprosthetic system 200, can be fluidly coupled with the lumen 504 of the portal expandable component 500. For instance, as shown in FIG. 2, the branch vessel expandable component 300 is coupled with the portal expandable component 500 via portal 516. While the portal 516 may include an aperture in the wall 510 of the portal expandable component 500, in some other examples, the portal 516 may include an alternative configuration.

For instance, in some examples, the wall 510 includes a recessed portion 520 that is recessed relative to the outer surface 514 of the wall 510 and positioned between the first and second ends 506 and 508 of the main body 502. In some such examples, the portal 516 is formed as an opening 518 in the recessed portion 520, as shown in FIG. 5A.

In some examples, the portal 516 may include a reinforced configuration. For instance, in some examples, the portal 516 may include one or more support walls, such as support walls 522. A support wall can have any preferred length, diameter, wall thickness or secondary lumen shape, such as an oval, polygon or “D shape”. In some examples, support walls can incorporate a support member such as a stent, as shown. Additionally or alternatively, a support wall can incorporate a support wall to branch member attachment feature such as a hook anchor, flared stent apex, or other securing means commonly known in the art. As shown in FIG. 5A, the support wall 522 extends from each opening 518 toward one of the first and second ends 506 and 508 of the main body 502. As such, the support wall 522 forms a secondary lumen 524, which is configured to receive one or more auxiliary expandable components of the modular endoprosthetic system 200, such as the branch vessel expandable component 300.

While the portal expandable component 500 shown in FIG. 5A includes a single portal 516, it is to be appreciated that the portal expandable component 500 may include multiple portals 516, including, for example, multiple support walls 522 and secondary lumens 524, where the multiple support walls can be oriented in generally the same direction, or in generally conflicting (non-parallel) directions relative to the longitudinal axis of the portal expandable component 500. For instance, in some examples, a first support wall of a first portal may extend toward the first end 506 of the portal expandable component 500, while a second support wall of a second portal extends toward the second end 508 of the portal expandable component 500. In one such example, a first support wall and secondary lumen having a first orientation will therefore define a first blood flow direction.

A “blood flow direction” is defined as the direction defined by the blood flow as it enters into the secondary lumen defined by the support wall. Conversely, a second support wall and secondary lumen having a second, different orientation will therefore define a second blood flow direction different from the first blood flow direction. The first and second blood flow directions can, if desired, be oriented between 0° and 180° from each other as desired. Further details on internal support walls and portal configurations for supporting branch members extending through openings or portals in the main body of an expandable endoprosthesis are disclosed in U.S. Pat. Nos. 6,645,242 and 9,314,328.

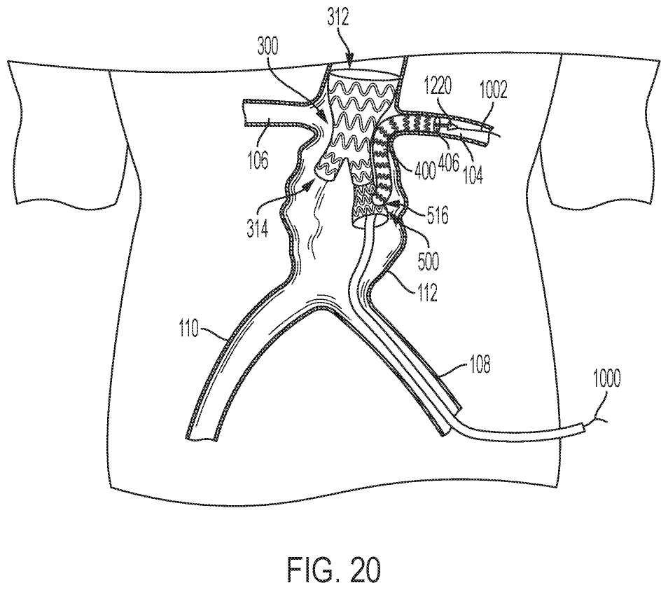

FIG. 5B illustrates the portal expandable component of FIG. 5A with the branch vessel expandable component 400 coupled therewith via the portal 516 (consistent with the configuration shown in FIG. 20). As shown, with the branch vessel expandable component 400 coupled with the portal expandable component 500 via the portal 516, the lumens of the branch vessel expandable component 400 and the portal expandable component 500 are fluidly coupled together.

As mentioned above, in some examples, the portal expandable component may include a plurality of portals for receiving respective branch vessel components therethrough for directing a portion of blood flow from the lumen of the portal expandable component to branch vessels. Such branch portals may be arranged in pairs facing in the same or in opposite directions (e.g., such as in a proximal direction, a distal direction, radially outwardly facing, any angles relative to the lumen axis, or any combination thereof).

The delivery systems and methods in accordance with various embodiments disclosed herein can utilize removable guidewire tubes to help facilitate guidewire cannulation therethrough subsequent to compacting the expandable implant toward a delivery configuration for endoluminal delivery to the treatment site. Such removable guidewire tubes may extend through main lumens of the expandable components, branch lumens of the expandable components, and/or portals of the expandable components. As shown in FIG. 6, for example, a first removable guidewire tube 1100 can be inserted through the portal 516 of the portal expandable component 500. Opposite ends 1102 and 1104 of the removable guidewire tube 1100 may extend axially beyond the first and second ends 506 and 508 of the portal expandable component 500. Removable guidewire tube can comprise the same materials mentioned herein for the catheter materials. Further details of materials and general construction of removable guidewire tubes are described in U.S. Pat. No. 8,273,115 to Hamer et al.

FIG. 7 shows the portal expandable component 500 in a compacted delivery configuration with a constraining sheath 1200. As shown, the portal expandable component 500 is coupled to and supported on a delivery catheter 1300. The constraining sheath 1200 extends over and releasably constrains the portal expandable component 500 toward the compacted delivery configuration. The constraining sheath 1200 can be removed from the portal expandable component according to known methods. Further details of materials and general construction of constraining sleeves can be found in U.S. Pat. No. 6,352,561 to Leopold et al.

As shown in FIG. 7, in the compacted delivery configuration, opposite ends 1102 and 1104 of the removable guidewire tube 1100 extend beyond respective opposite ends 1202 and 1204 of the constraining sheath 1200 to allow a guidewire to be routed through the portal 516 of the portal expandable component 500 via the removable guidewire tube 1100 even though the portal expandable component 500 is radially inwardly compressed toward or otherwise covered while in the delivery configuration by the constraining sheath 1200. In various embodiments, methods of endoluminally delivering a modular endoprosthesis can include inserting a first guidewire into the vasculature through an access site and into the vasculature to be treated.

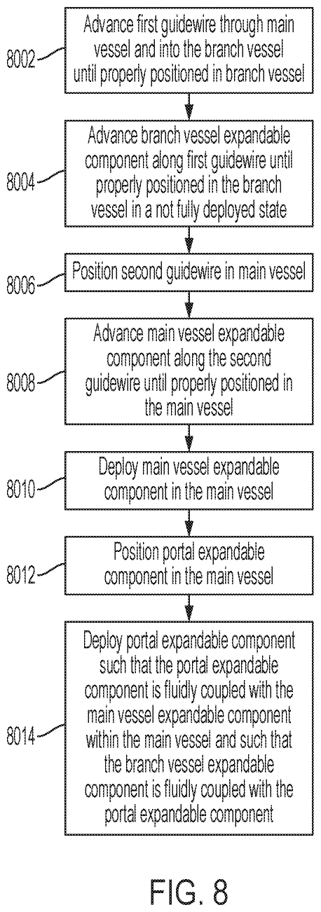

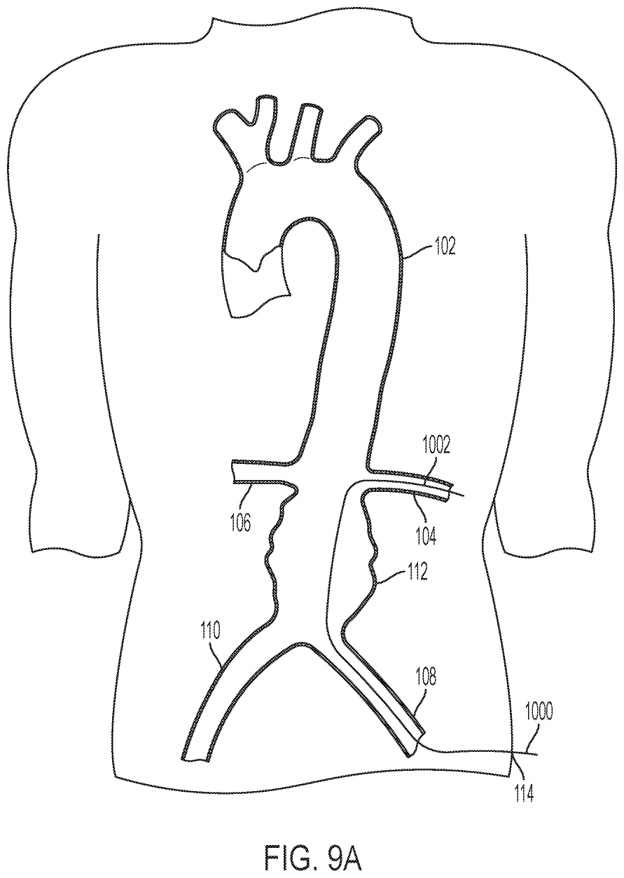

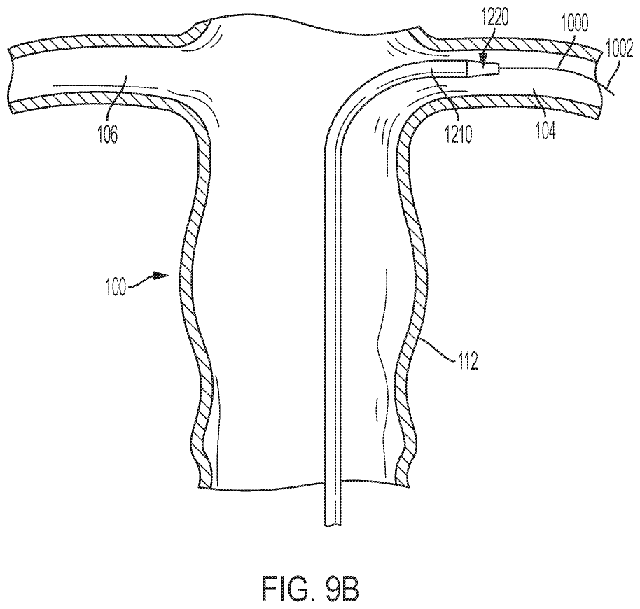

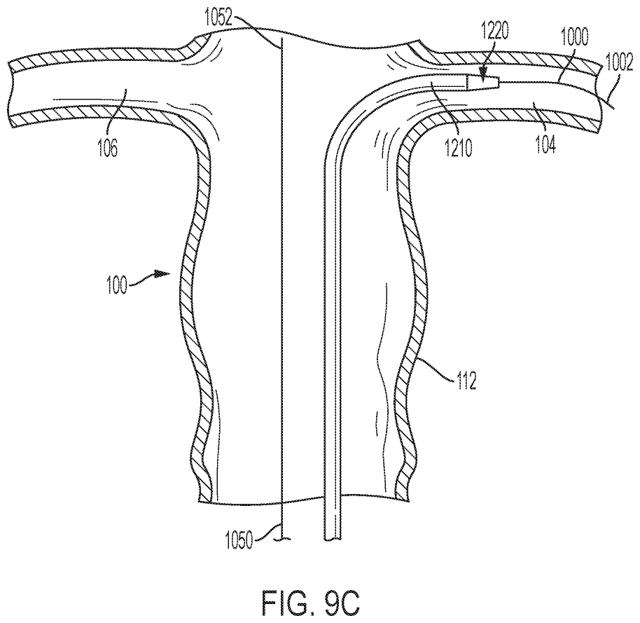

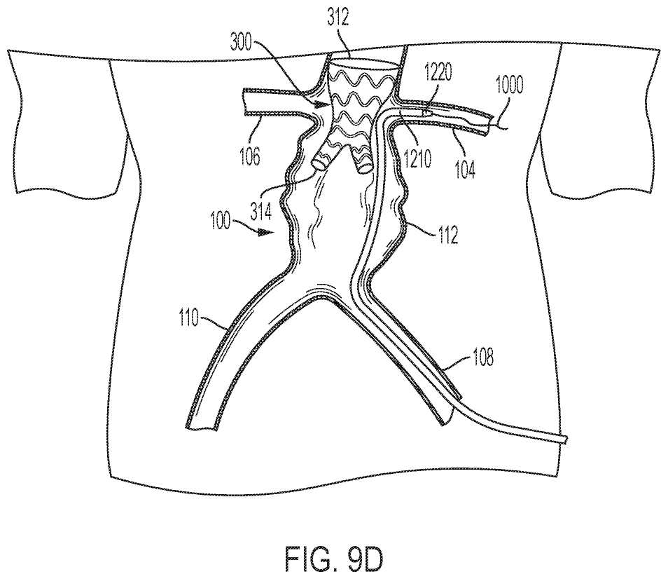

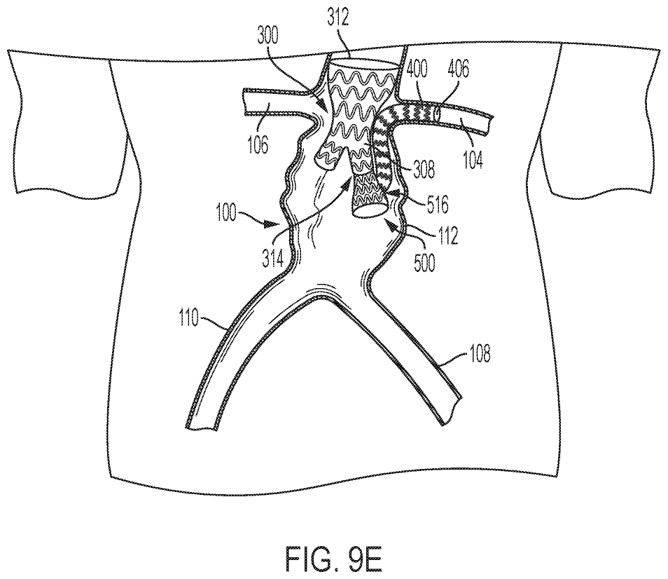

FIG. 8 is a flow chart illustrating a method for endoluminally delivering a modular endoprosthesis in accordance with the present disclosure. As shown in FIG. 8, at step 8002 a first guidewire is advanced through the main vessel and into a branch vessel. FIG. 9A shows a first guidewire 1000 that has been inserted into the femoral artery through an access site 114, routed through one of the iliac arteries 108, into the descending aorta, and into a first branch vessel 104. As shown in FIG. 9A, a first end 1002 of the first guidewire 1000 is positioned within the first branch vessel 104 while the second end 1004 of the first guidewire 1000 extends to a position exterior to the patient. As such, the second end 1004 is accessible from outside the patient's body and can be used to delivery subsequent components of the modular endoprosthetic system 200, as described further below. The positioning of the first end 1002 of the first guidewire within the first branch vessel 104 is intended to be for example purposes only, and should not be construed as limiting. As such, it is to be appreciated that the first end 1002 of the first guidewire 1000 may positioned within any branch vessel from a main vessel in the patient's anatomy, and is not limited to the branch vessels of the aorta. It will also be appreciated that the first guidewire 1000 may be introduced to the anatomy and advanced to the branch vessel according to known methods. The first guidewire 1000 is shown in FIG. 9 as being advanced in a retrograde direction (i.e., against the flow of blood, also referred to as being advanced “upstream”).

Referring back to FIG. 8, at step 8004, a branch vessel expandable component is advanced along the first guidewire until it is properly positioned within the branch vessel. The branch vessel expandable component is generally delivered by advancement along the first guidewire in a radially constrained or compacted delivery configuration, as mentioned above. As shown in FIG. 9B is a detail view of a section of the patient's anatomy with the branch vessel expandable component in a radially constrained and compacted delivery configuration (e.g., via a constraining sheath 1210), where the branch vessel expandable component 400 is disposed about a delivery member 1220 (e.g., a catheter). In various examples, proper positioning of the branch vessel expandable component 400 occurs where at least a portion of the branch vessel expandable component 400 is positioned within the branch vessel 104 such that, upon expansion of the branch vessel expandable component 400 to a delivered configuration, the branch vessel expandable component 400 is operable to engage the branch vessel (e.g., the vessel or tissue wall) to maintain a coupling between the branch vessel expandable component 400 and the branch vessel 104.

Referring back to FIG. 8, at step 8006, a second guidewire is positioned within the main vessel. In various examples, the second guide wire is positioned within the main vessel in a region proximate the branch vessel within which the branch vessel expandable component is positioned. For instance, as shown in FIG. 9C, a second guidewire 1050 is positioned within the main vessel 100. As shown, the second guidewire 1050 is positioned such that an end 1052 of the second guidewire 1050 is upstream of the branch vessel 104 within which the branch vessel expandable component 400 is positioned.

Referring back to FIG. 8, at steps 8008 and 8010, a main vessel expandable component is advanced along the second guidewire to a position within the main vessel, and subsequently deployed, once properly positioned. In various examples, proper positioning of the main vessel expandable component involves positioning the main vessel expandable component such that an end of the main vessel expandable component lands or otherwise engages the main vessel upstream from the branch vessel within which the branch vessel expandable component is positioned. Such a configuration provides that the branch vessel expandable component does not interfere with the end of the main vessel expandable component engaging the vessel wall or tissue about a periphery of the end of the main vessel expandable component, thereby allowing for the main vessel expandable component to seal against the main vessel wall without interference from other expandable components. FIG. 9D shows the main vessel expandable device 300 deployed within the main vessel 100 such that the proximal end 312 of the main vessel expandable device 300 engages the vessel wall of the main vessel upstream from the branch vessel 104. Additionally, as shown in FIG. 9D, the distal end 314 of the main vessel expandable component 300 is positioned downstream of the branch vessel 104.

Referring back to FIG. 8, at steps 8012 and 8014 the portal expandable component is positioned within the main vessel and deployed such that the portal expandable component is fluidly coupled with the main vessel expandable component and such that the branch vessel expandable component is fluidly coupled with the portal expandable component. FIG. 9E shows the portal expandable component 500 in a deployed configuration where the portal expandable component 500 is fluidly coupled with the main vessel expandable component 300 (e.g., via leg 308) and where the portal expandable component 500 is fluidly coupled with the branch vessel expandable component 400 (e.g., via the portal 516) such that the main vessel expandable component 300 is fluidly coupled with the branch vessel expandable component 400. In the configuration illustrated in FIG. 9E, the portal expandable component 500 is coupled with the main vessel expandable component 300 such that the portal 516 is positioned downstream of the branch vessel 104. As shown, the first end 506 of the portal expandable component 500 deployed within a lumen of the first leg 308 of the main vessel expandable component 300 at the distal end 314 of the main vessel expandable component 300. With the portal 516 positioned downstream to the branch vessel 104 within which the branch vessel expandable component 400 is positioned, blood flow to the branch vessel via the branch vessel expandable component 400 occurs retrograde.

It is to be appreciated that a similar method to the above may be implemented to deliver and deploy corresponding expandable components to the branch vessel 106 (see, e.g., FIG. 2 for an example involving multiple branch vessels). In some examples involving multiple branch vessels (e.g., renal arteries) it is to be appreciated that branch vessel expandable components are positioned within each of the branch vessels prior to deployment of the main vessel expandable component.

As indicated above, at step 8014, the portal expandable component is deployed such that the portal expandable component is fluidly coupled with the main vessel expandable component and such that the branch vessel expandable component is fluidly coupled with the portal expandable component. In some examples, the branch vessel expandable component is fluidly coupled with the portal expandable component via a bridge expandable component. The portal expandable component 500 may be coupled with the main vessel expandable component 300 according to known methods. Similarly, the branch vessel expandable component 400 may be coupled with the portal expandable component 500 according to known methods.

As shown in FIG. 2, the bridge expandable component 600 is positioned between the branch vessel expandable component 400 and the portal expandable component 500. Thus, in various examples, the bridge expandable component 600 is configured to engage with each of the branch vessel expandable component 400 and the portal expandable component 500. The bridge expandable component 600 has a construction consistent with the branch vessel expandable component 400 described above, including, for example, a support component, a graft component, and first and second ends with a lumen extending therethrough. As shown, the bridge expandable component 600 is configured to be delivered to the treatment site and deployed with a first end coupled with the portal 516 of the portal expandable component 500 and with the second end coupled with the branch vessel expandable component 400. In some examples, the branch vessel expandable component 400 includes one or more tissue anchors for engaging the tissue (e.g., vessel wall) of the branch vessel 104.

In some examples, the bridge expandable component 600 is configured to be deployed at least partially within a lumen of the branch vessel expandable component 400 and at least partially within the portal expandable component 500. In some such examples, the bridge expandable component 600 extends through the portal 516 such that the bridge expandable component 600 is fluidly coupled with the portal expandable component 500.

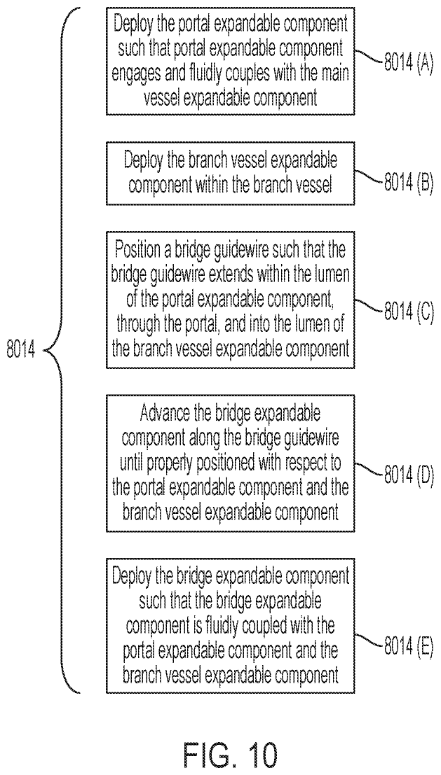

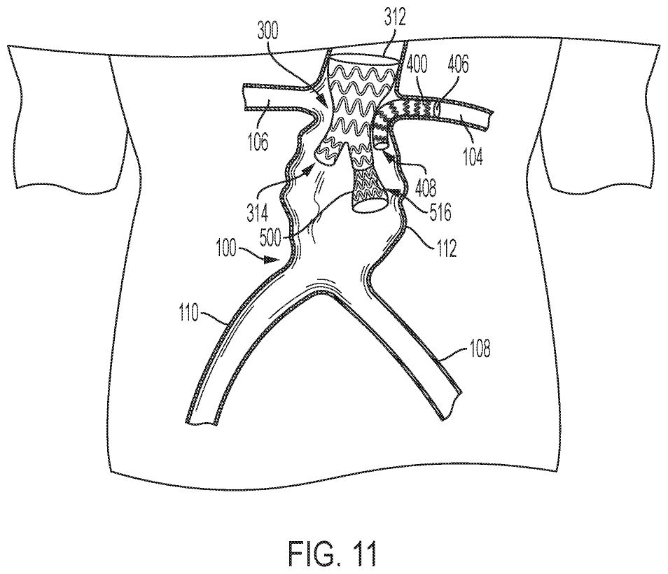

FIG. 10 illustrates a flow chart outlining an example method consistent with step 8014 of FIG. 8 for fluidly coupling the branch vessel expandable component 400 and the portal expandable component 500 via the bridge expandable component 600. At step 8014(A), the portal expandable component is deployed such that the portal expandable component engages and fluidly couples with the main vessel expandable component. FIG. 11 shows a portal expandable component 500 deployed such that the portal expandable component 500 is engaged and fluidly coupled with the main vessel expandable component 300 consistent with the discussion above regarding FIG. 9E. As shown, the portal expandable component 500 is deployed such that the portal 516 is positioned downstream relative to the branch vessel 104. In some examples, the portal expandable component 500 is positioned at least partially inside the gate of the main vessel expandable component 300 (e.g., in an overlapping relationship with at least one of the legs 308 and 310 of the main vessel expandable component 300) such that, upon deployment of the portal expandable component 500, the portal 516 will be fluidly accessible/coupled with the lumen of the main vessel 100.

Referring again to FIG. 10, at step 8014(B), the branch vessel expandable component is deployed. The branch vessel expandable component may be deployed prior to or after deploying the portal expandable component. FIG. 11 shows the branch vessel expandable component 400 in the deployed configuration. As shown, the branch vessel expandable component 400 is deployed such that the first end 406 of the branch vessel expandable component 400 is positioned within the branch vessel and such that the second end 408 is positioned within the main vessel. As such, the portal expandable component 500 is deployed with the portal 516 positioned downstream relative to the first end 406 of the branch vessel expandable component 400. In some examples, the branch vessel expandable component 400 may be deployed such that the first and second ends 406 and 408 of the branch vessel expandable component 400 are positioned within the branch vessel.

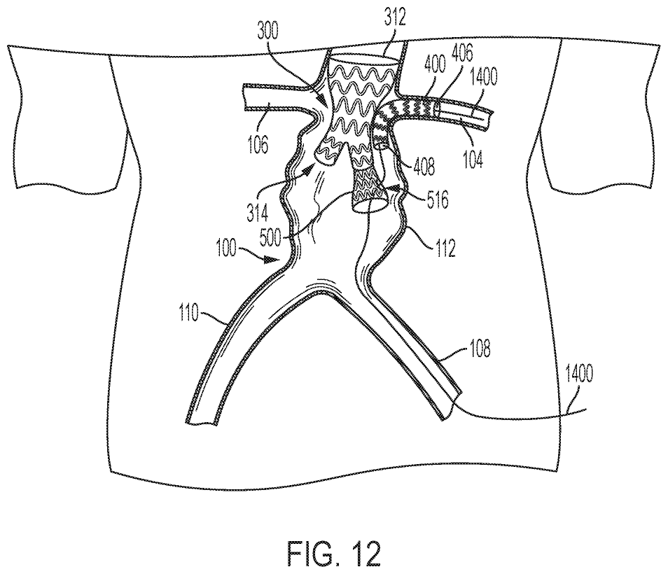

Referring again to FIG. 10, at step 8014(C), a bridge guidewire is positioned such that the bridge guidewire extends within the lumen of the portal expandable component, through the portal, and into the lumen of the branch vessel expandable component. FIG. 12 shows a bridge guidewire 1400 extending from outside the patient's body, into the main vessel 100, into the lumen of the portal expandable component 500, through the portal 516, and into the lumen of the branch vessel expandable component 400.

Referring again to FIG. 10, at steps 8014(D) and 8014(E), a bridge expandable component is advanced to a position that extends between the portal expandable component and the branch vessel expandable component and deployed such that the bridge expandable component is fluidly coupled with the portal expandable component and the branch vessel expandable component. The bridge expandable component 600 may be coupled with the branch vessel expandable component 400 and the portal expandable component 500 according to known methods.

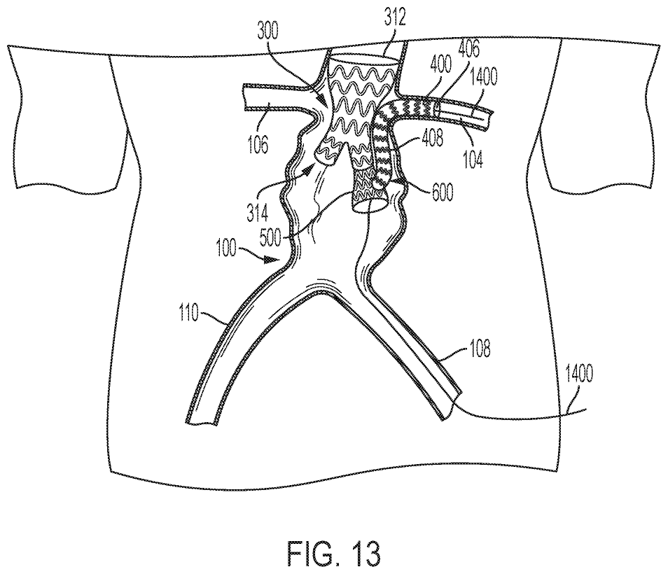

FIG. 13 shows a bridge expandable component 600 that fluidly couples the portal expandable component 500 and the branch vessel expandable component 400. As shown, the bridge expandable component 600 extends from the portal expandable component 500 through the portal 516, and couples with the branch vessel expandable component 400 proximate the second end 408 of the branch vessel expandable component 400. In some examples, the bridge expandable component 600 is deployed such that a portion of the bridge expandable component 600 is positioned within the lumen of the branch vessel expandable component 400. With the main vessel expandable component 300, the branch vessel expandable component 400, the portal expandable component 500, and the bridge expandable component 600 so configured, the modular endoprosthetic system 200 provides that blood flow can be supplied to the branch vessel 104 via retrograde flow through the branch vessel expandable component 400 and the bridge expandable component 600. As mentioned above, blood propagates through the main vessel expandable component 300 and the portal expandable component 500 via antegrade flow. Thus, it is to be appreciated that the modular endoprosthetic system 200 is configured such that blood flow therethrough may be antegrade in a first region and retrograde in a second region.

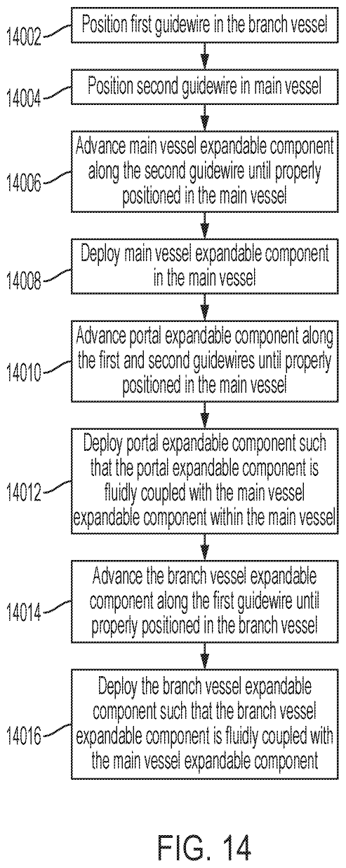

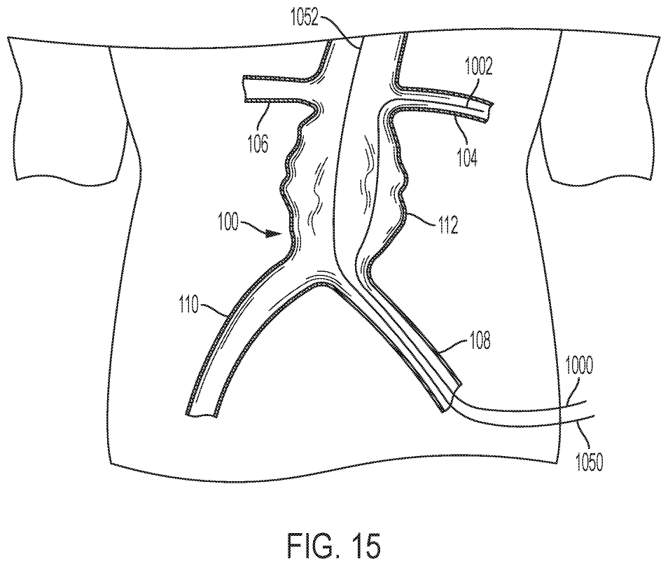

FIG. 14 illustrates a flow chart outlining another example method for delivering the modular endoprosthesis of the present disclosure. At steps 14002 and 14004, first and second guidewires are positioned in the branch and main vessels, respectively. In some examples, the first guidewire is positioned in the branch vessel prior to the second guidewire being positioned in the main vessel, while in other examples, the first guidewire is positioned in the branch vessel after the second guidewire is positioned in the main vessel. FIG. 15 shows first and second guidewires 1000 and 1050 positioned in the branch and main vessels, 104 and 100, respectively.

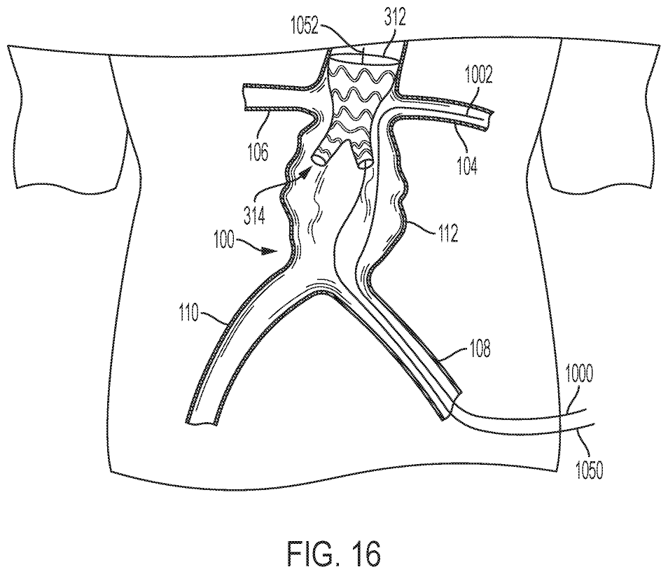

Referring again to FIG. 14, at steps 14006 and 14008 the main vessel expandable component is advanced along the second guidewire 1050 to a position within the main vessel, and subsequently deployed, once properly positioned. In some examples, steps 14006 and 14008 correspond with steps 8008 and 8010. Thus, as explained above, in various examples, proper positioning of the main vessel expandable component involves positioning the main vessel expandable component such that an end of the main vessel expandable component lands or otherwise engages the main vessel upstream from the branch vessel, which provides that the branch vessel expandable component does not interfere with the end of the main vessel expandable component engaging the vessel wall or tissue about a periphery of the end of the main vessel expandable component, thereby allowing for the main vessel expandable component to seal against the main vessel wall without interference from other expandable components. FIG. 16 shows the main vessel expandable device 300 deployed within the main vessel 100 such that the proximal end 312 of the main vessel expandable device 300 engages the vessel wall of the main vessel upstream from the branch vessel 104 (see, e.g., the discussion above regarding deployment of the main vessel expandable device 300). Additionally, as shown in FIG. 16, the distal end 314 of the main vessel expandable component 300 is positioned downstream of the branch vessel 104. The proximal end 312 of the main vessel expandable device 300 may employ known means to engage the vessel wall of the main vessel, including anchors such as barbs.

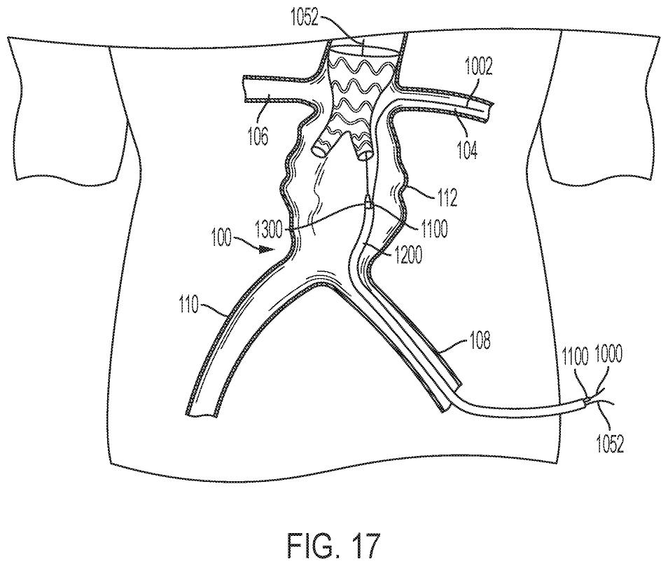

Referring again to FIG. 14, at step 14010 the portal expandable component is advanced along each of the first and the second guidewires 1000 and 1050 until it is properly positioned within the main vessel relative to the main vessel expandable device. FIG. 17 shows the portal expandable component 500 (concealed from view in FIG. 17 by constraining sheath 1200) in a constrained and compacted delivery configuration consistent with the discussion above. As shown, the portal expandable component 500 is advanced along the first and second guidewires 1000 and 1050, where the first guidewire 1000 extends through the portal 516 (which is concealed from view in FIG. 17 by constraining sheath 1200) and the lumen of the portal expandable component 500. In the example illustrated in FIG. 17, the first guidewire 1000 extends through a removable guidewire tube 1100 that extends through the portal and the lumen of the portal expandable component 500. However, as mentioned above, the removable guidewire tube is not required and is therefore optional and not essential. Additionally, the second guidewire 1050 extends through the lumen of the portal expandable component 500 without extending through the portal 516 (e.g., extends through the lumen from the first end 506 to the second end 508 of the portal expandable component 500.

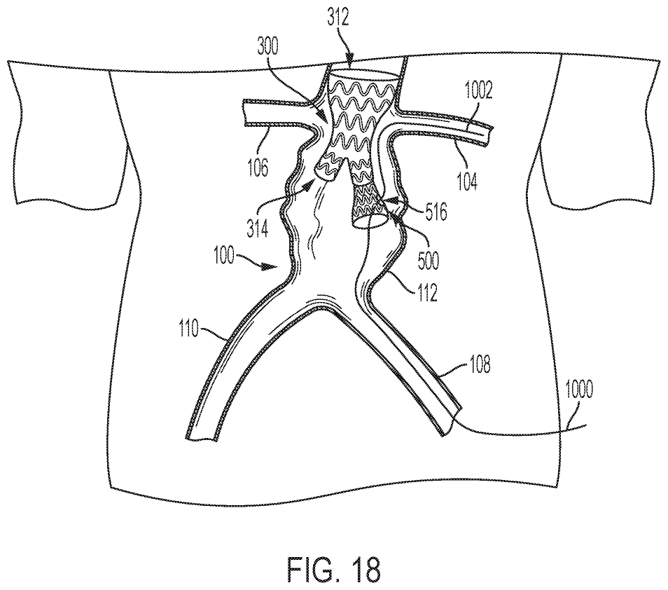

Referring again to FIG. 14, at step 14012, once properly positioned, the portal expandable component is deployed such that the portal expandable component is fluidly coupled with the main vessel expandable component. FIG. 18 shows the portal expandable component 500 is deployed, such that the portal expandable component 500 is fluidly coupled with the main vessel expandable component 300 with the portal 516 positioned downstream from the branch vessel 104. The portal expandable component 500 is deployed and coupled with the main vessel expandable component 300 consistent with the discussions above (see, e.g., the discussion regarding FIGS. 9E and 11). Additionally, as shown in FIG. 18, the first guidewire 1000 extends into the lumen of the portal expandable component 500 and out through the portal 516, and into the branch vessel 104.

In some examples, after the portal expandable component 500 is deployed, the removable guidewire tube 1100 may be removed from the portal expandable component 500 along the first guidewire 1000, leaving behind the first guidewire for future use, as described in greater detail below. Alternatively, in some examples, the removable guidewire tube 1100 can be removed after loading the portal expandable component 500 on the first guidewire 1000 and prior to advancing the portal expandable component 500 into the patient's vasculature (e.g., used for pre-cannulation only). That is, the removable guidewire tube 1100 may be used to load the portal expandable component 500 and its delivery member on the first guidewire 1000, but may be removed prior to advancing the same within the vasculature of the patent. In some examples, the removable guidewire tube 1100 may be removed by the physician by grasping the second end 1104 and withdrawing the removable guidewire tube 1100 in a direction opposite that for advancing the removable guidewire tube 1100 into the vasculature (e.g., distally relative to a lower access site).

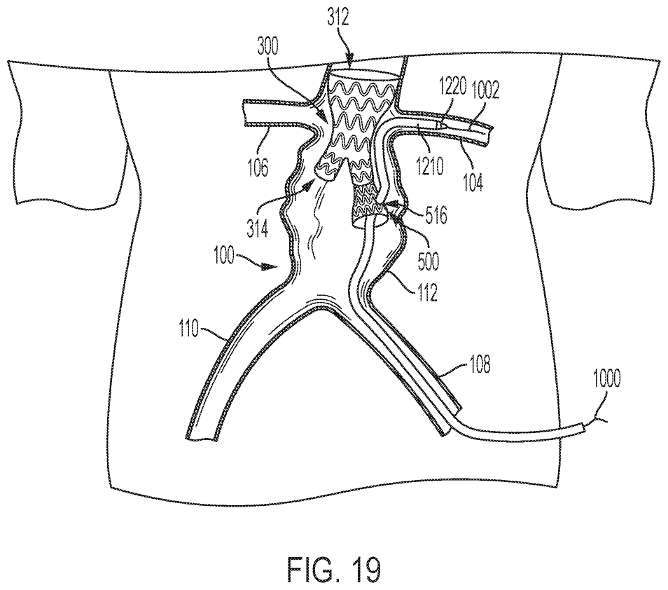

Referring again to FIG. 14, at step 14014, the branch vessel expandable component is advanced along the first guidewire until properly positioned with respect to the portal expandable component and the branch vessel. In various examples, the branch vessel expandable component is advanced while maintained in a constrained and collapsed delivery configuration (see discussion above). Additionally, in various examples, proper positioning of the branch vessel includes positioning of the branch vessel expandable component such that, upon deployment, the branch vessel expandable component will engage the branch vessel and the portal expandable component such that the branch vessel expandable component fluidly couples together the branch vessel and the portal expandable component. FIG. 19 shows the branch vessel expandable component 400 being advanced along the first guidewire 1000. As shown, the branch vessel expandable component 400 (concealed from view by constraining sheath 1210) is coupled with the delivery catheter 1220 which has been advanced along the first guidewire 1000 to the position shown such that the branch vessel expandable component 400 can be deployed, at least in part, with a portion thereof within the branch vessel. Conventional fluoroscopy techniques utilizing radiopaque markers on any one or multiple components of the modular endoprosthetic system 200 and/or delivery members can be utilized to facilitate positioning of the various expandable components of the modular endoprosthetic system 200 at the treatment site. For example, radiopaque markers can be located at or near the ends of the expandable components, and/or at or near the portal(s) of the portal expandable component 500, and/or at or near or along the legs of the branched endoprosthesis, and/or at any position along the delivery members, to facilitate orientation and relative positioning between the expandable components of the modular endoprosthetic system 200 and those regions of the patient's anatomy to be treated. It is to be appreciated that radiopaque markers may be utilized in any of the components discussed herein.

Referring again to FIG. 14, at step 14016, with the branch vessel expandable component properly positioned relative to the branch vessel 104 and the portal expandable component 500, the branch vessel expandable component is deployed such that the branch vessel is fluidly coupled with the portal expandable component 500 via the branch vessel expandable component 400. FIG. 20 shows the branch vessel expandable component 400 in a fully deployed configuration where the branch vessel is fluidly coupled with the portal expandable component 500. As such, the branch vessel 104 can be supplied blood flowing through the modular endoprosthesis via retrograde flow through the branch vessel expandable component 400. That is, the branch vessel expandable component 400 provides that blood flowing through the main vessel expandable component 300 can enter the branch vessel 104 by flowing into the portal expandable component 500 and then into the branch vessel expandable component 400, where the blood flow through the portal expandable component 500 is antegrade flow and where the blood flow through the branch vessel expandable component 400 is retrograde flow. As mentioned above, it is to be appreciated that perfusion to the other renal artery 106 (see, e.g., FIG. 2) in addition to, or as an alternative to perfusion to renal artery 104, is accomplished via a second (or additional) portal expandable component 500 and a second (or additional) branch vessel expandable component 400, and optionally a bridge expandable component 600, where the delivery and deployment of such components is consistent with the discussion above but for accessing and deploying within the branch vessel 106 instead of the branch vessel 104.

In various examples, the catheters, introducer sheaths, hubs, handles and other components referred to herein and usable in the disclosed systems and methods can be constructed using any suitable medical grade material or combination of materials using any suitable manufacturing process or tooling. Suitable medical grade materials can include, for example, nylon, polyacrylamide, polycarbonate, polyethylene, polyform aldehyde, polymethylmethacrylate, polypropylene, polytetrafluoroethylene, expanded polytetrafluoroethylene, polytrifluorochlorethylene, polyvinylchloride, polyurethane, elastomeric organosilicon polymers, Pebax® polyether block amide, and metals such as stainless steels and nitinol. Catheters can also include a reinforcing member, such as a layer of metal braid.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

1. A method comprising:
providing a first expandable device configured to be deployed in a main vessel;
providing a second expandable device configured to interface with the first expandable device, wherein the second expandable device includes a portal therein;
providing a branch vessel expandable device configured once expanded to form a fluid connection between a branch vessel and the second expandable device through the portal;
placing a branch guidewire into the branch vessel;
positioning the branch vessel expandable device over the branch guidewire into the branch vessel while maintained in a not fully deployed state;
placing and deploying the first expandable device in the main vessel, wherein the branch vessel expandable device is positioned exterior to the first expandable device;
placing and deploying the second expandable device downstream to the branch vessel, wherein the branch guidewire and the branch vessel expandable device each extend through the portal of the second expandable device; and
deploying the branch vessel expandable device such that the branch vessel expandable device is fluidly coupled with the second expandable device via the portal of the second expandable device,
wherein blood is perfused into the branch vessel through retrograde flow.

2. The method of claim 1, wherein the branch guidewire is placed into a renal artery, and wherein the branch vessel expandable device is placed over the branch guidewire into the renal artery while maintained in the not fully deployed state, and wherein the first expandable device is placed and deployed in an aorta of a patient with the branch vessel expandable device positioned exterior to the first expandable device, and wherein the second expandable device is placed and deployed at least partially downstream to the renal artery with the branch guidewire and the branch expandable device extending through the portal to form a fluid connection between the renal artery and the second expandable device to provide for retrograde perfusion of blood to the renal artery.

3. The method of claim 1, wherein the main vessel is a common iliac artery and wherein the branch vessel is an internal iliac artery.

4. The method of claim 1, wherein the main vessel is an external iliac artery and wherein the branch vessel is a femoral artery.

5. The method of claim 1, wherein positioning the branch vessel expandable device over the branch guidewire into the branch vessel includes advancing the branch vessel expandable device over the branch guidewire after the second expandable device is deployed.

6. The method of claim 5, wherein the branch vessel expandable device is deployed after the second expandable device is deployed.

7. The method of claim 1, wherein the branch vessel expandable device is directly coupled to the second expandable device.

8. A method comprising:
providing a first expandable device configured to be deployed in a blood vessel;
providing a second expandable device configured to interface with the first expandable device, wherein the second expandable device includes a portal therein;
providing a branch expandable device configured to form a fluid connection between a branch vessel and the second expandable device through the portal;
placing a branch guidewire into the branch vessel;
placing and deploying the first expandable device in the main vessel;
placing and deploying the second expandable device downstream from the branch vessel, wherein the branch guidewire extends through the portal;
positioning the branch expandable device over the branch guidewire to interconnect the branch vessel and the second expandable device exterior to the first expandable device; and
deploying the branch expandable device to form a fluid connection between the branch vessel and the second expandable device,
wherein blood is perfused into the branch vessel through retrograde flow.

9. The method of claim 8, wherein the branch guidewire is placed into a renal artery, and wherein the second expandable device is placed and deployed at least partially downstream to the renal artery with the second expandable device fluidly coupled with the renal artery via the branch expandable device to provide for retrograde perfusion of blood to the renal artery.

10. The method of claim 8, wherein the main vessel is a common iliac artery and wherein the branch vessel is an internal iliac artery.

11. The method of claim 8, wherein the main vessel is an external iliac artery and wherein the branch vessel is a femoral artery.

12. The method of claim 8, further comprising deploying a third expandable device between the second expandable device and the branch vessel expandable device.

13. The method of claim 12, wherein the third expandable device is advanced into position over the branch guidewire.

14. The method of claim 12, wherein the third expandable device is deployed after the branch vessel expandable device is deployed and after the second expandable device is deployed.

15. The method of claim 1, wherein the second expandable device is provided in a collapsed delivery configuration with a removable guidewire tube extending through the portal to allow for insertion of the branch guidewire therethrough.

16. The method of claim 15, further comprising removing the removable guidewire tube after insertion of the second guidewire through the removable guidewire tube.

17. The method of claim 16, further comprising removing the removable guidewire tube prior to insertion of the second expandable device into the main vessel.

18. The method of claim 1, wherein the first and second expandable devices are deployed prior to deploying the branch vessel expandable device.

19. The method of claim 8, wherein the branch vessel expandable device is deployed prior to the second expandable device being deployed.

20. The method of claim 1, wherein the branch vessel expandable device is deployed after the second expandable device is deployed.

21. The method of claim 1, wherein the second expandable device is deployed after the first expandable device is deployed.

22. The method of claim 1, wherein each of the first expandable device, the second expandable device, and the branch expandable device are advanced from a first access site that is downstream from the branch vessel.

23. The method of claim 1, wherein the branch vessel expandable device is fluidly coupled to the second expandable device via the portal.

24. The method of claim 1, wherein the first expandable device is advanced over a first guidewire separate distinct from the branch guidewire.

25. The method of claim 24, wherein the second expandable device is advanced over each of the first guidewire and the branch guidewire.

26. The method of claim 1, wherein the portal is positioned in a sidewall of the second expandable device.

27. An expandable device configured to repair a main vessel extending from an upstream end to a downstream end, the expandable device comprising:
a first expandable device configured to be deployed in a blood vessel;
a second expandable device configured to interface with the first expandable device and including a portal in a sidewall of the second expandable device; and
a branch vessel expandable device configured to form a fluid connection between a branch vessel and the second expandable device by extending through the portal,
wherein the branch expandable device is configured to have sufficient length to allow for retrograde perfusion to the branch vessel through the branch vessel expandable device in association with the second expandable device being implanted downstream from the branch vessel.

28. The expandable device of claim 27, wherein the branch vessel expandable device is configured with sufficient radial expansion force to maintain significant flow therethrough when deployed exterior to the first expandable device between the first expandable device and a wall of the main vessel.

29. The expandable device of claim 27, wherein the branch vessel expandable device is directly coupled to the second expandable device.

30. The expandable device of claim 27, further comprising:
a third expandable device extending between the second expandable device and the branch vessel expandable device and configured to allow for retrograde perfusion to the branch vessel through the branch vessel expandable device and the third expandable device.

31. The expandable device of claim 27, wherein the side wall of the second expandable device comprising a recessed portion that is recessed relative to the side wall, the portal being located in the recessed portion.

32. The expandable device of claim 27, further comprising a downstream expandable device extending from the second expandable component to fluidly couple the second expandable component to one or more vessels downstream of the second expandable component.

33. The expandable device of claim 27, wherein the first expandable device includes a body portion, a first leg and a second leg branching from the body portion, and the second expandable device is configured to interface with one of the first leg and the second leg of the first expandable device.

34. The expandable device of claim 33, wherein the first leg and the second leg are structurally biased to angle apart from one another.

35. The expandable device of claim 33, wherein the second expandable device is configured to interface with the first second leg of the first expandable device, and further comprising an additional second expandable device configured to interface with the second leg of the first expandable device and including a portal in a sidewall of the second expandable device.

36. The expandable device of claim 35, further comprising an additional branch vessel expandable device configured to form a fluid connection between a second branch vessel and the additional second expandable device by extending through the portal.

37. The expandable device of claim 27, wherein the second expandable component includes a proximal end and a distal end, and a tapered configuration with the proximal end having a diameter less than the distal end.

38. The expandable device of claim 27, wherein the portal of the second expandable device is an aperture in the sidewall of the second expandable device.

39. The expandable device of claim 27, further comprising a bridge expandable component configured to position between the second expandable device configured and the first expandable device.

40. The expandable device of claim 39, wherein the bridge expandable component is configured to deploy with a first end coupled with the portal of the second expandable component and with a second end coupled with the second expandable device component.

41. The expandable device of claim 40, wherein the branch vessel expandable component includes one or more tissue anchors for engaging tissue.
